(19) United States
(12) Patent Application Publication
(10) Pub. No.: US 2014/0275772 A1
(43) Pub. Date: Sep. 18, 2014

(54) INTUBATION DEVICE WITH VIDEO AND ANATOMIC STYLET STEERING

(71) Applicant: Robert Michael Chuda, Yonkers, NY (US)

(72) Inventor: Robert Michael Chuda, Yonkers, NY (US)

(21) Appl. No.: 14/294,128

(22) Filed: Jun. 2, 2014

Related U.S. Application Data
(63) Continuation-in-part of application No. 12/058,492, filed on Mar. 28, 2008, now abandoned.
(60) Provisional application No. 60/920,539, filed on Mar. 29, 2007.

(51) Int. Cl. A61M 16/04; A61B 1/267

(52) U.S. Cl. CPC A61M 16/0488; A61B 1/267
USPC 600/104

(57) ABSTRACT

A one piece, one step, steerable video intubation device has an intubation device body having a downwardly extending stylet, a proximal handle, and a video display. The flexible stylet has firming and bending tendons controlled by a trigger for controlled anatomic shaping and anatomically accurate steerage of the stylet. The stylet is inserted into an endotracheal tube for an intubation procedure, or other tubes for insertion into various body orifices. The stylet and mounted endotracheal tube are guided by a video image from a camera and light on the distal tip end of the stylet. The intubation device is removed, with the endotracheal or other surgical tube remaining in proper place in the patient. Sterility is maintained via a disposible sheath or condom placed over the stylet during use.

### CROSS-REFERENCE TO RELATED APPLICATIONS

The present utility patent application is a continuation-in-part of applicant's utility patent application Ser. No. 12/058,492, filed Mar. 28, 2008 which claims the benefit of provisional application No. 60/920,539, filed Mar. 29, 2007.

### STATEMENT REGARDING FEDERALLY SPONSORED RESEARCH OR DEVELOPMENT

Not applicable.

### THE NAMES OF THE PARTIES TO A JOINT RESEARCH OR DEVELOPMENT

Not applicable.

### BACKGROUND OF THE INVENTION

1. Field of the Invention

The present invention relates to medical devices useful to aid the intubation, or placing of tubes, in the accessible body cavities of patients. More particularly, the preferred embodiment of the present invention is a device to aid in the placement of endotracheal tubes (breathing tubes) such that ventilation and oxygenation of the lungs can be accomplished. Even more particularly, the present invention is designed to aid intubation procedures in the setting of anatomic or other difficult conditions which might prevent intubation from being successfully performed using routine equipment and methods.

2. Description of Related Art Including Information Disclosed Under 37 CFR 1.97 and 1.98

Currently, the majority of endotracheal intubation procedures are effected by the use of a rigid direct laryngoscope inserted through the mouth. The endotracheal intubation procedure involves passing a tube via the mouth or nasal passages through the pharynx (area at the back of the mouth and nose), past the tongue and epiglottis, and through the vocal cords (glottis) into the trachea. This tube is then used to maintain the airway and support ventilation of the lungs, a critical life support function. Using a straight or curved blade with a light along its length, the tongue and epiglottis are lifted out of the way allowing direct visualization of the vocal cords such that an endotracheal tube can be advanced into the trachea.

In cases where direct visualization of the opening to the larynx is not possible, due to anatomic variation, trauma, or pathologic process, the procedure may be difficult causing injury or even failure to be successfully performed. Conditions such as increased hypopharyngeal tissue as found in morbid obesity. short muscular neck, cephalad location of the larynx, prominent teeth, mandibular shortening, inability to open the mouth sufficiently, inability to position and manipulate the head and neck (such as cervical spine fixation or injury), tumors or masses of the oropharynx or larynx can all prevent direct laryngoscopy necessary for intubation with a direct laryngoscope.

If a difficult intubation for one of the foregoing reasons is anticipated in advance (elective or non-emergent situation), certain methods and devices would then be used to secure the airway. Intubation can be accomplished over a flexible fiber optic bronchoscope or fiber optic laryngoscope. These devices are expensive and very delicate, and require considerable expertise to use effectively. In the non-emergent case, frequently an expert with this equipment will be notified in advance, since typically neither the expert or the fiber optic scope are readily available on short notice. Also, the construction and flexibility of these devices makes them awkward, even in the expert's hands, especially for orotracheal intubation, as the typical flexing pattern of these types of scopes at times cannot navigate the angles necessary for orotracheal intubation and can only be used successfully via the nasal route. These fiber optic devices also suffer from degradation in visual transmission quality due to bending or breaking of the individual fiber optic bundles with repeated use. In between procedures, these devices must be formally processed for cleaning and sterilization, as they often come into direct contact with mucus membranes and have channels or conduits within the scope which become contaminated by trapping secretions.

This need for high level decontamination or sterilization processing typically removes the device from availability for use for a period of time, usually over an hour and perhaps even several hours or the entire day. Since these devices are delicate and expensive, most hospitals own a limited number of them, and down time during reprocessing can result in lack of availability of necessary equipment in emergency situations. After sterilization, they are typically stored with other emergency and difficult airway management devices in a central location or on a moveable ‘airway cart’ found in many operating rooms and other anesthetizing locations. Fiber optic intubation in the conscious, spontaneously breathing patient is considered the gold standard of securing the airway in patients that cannot be intubated via direct laryngoscopy, but is a difficult and time consuming process requiring considerable training and expertise.

However, this technique of conscious fiber optic intubation can rarely be used in the setting of unanticipated difficult intubation, such as when direct laryngoscopy fails during induction of general endotracheal anesthesia and the patient has received paralyzing drugs necessary as surgical muscle relaxants and the anesthesiologist must breath for the patient immediately. This crisis situation, known as CICV (Can't Intubate, Can't Ventilate) can have a fatal outcome. Numerous devices, such as specialized rigid laryngoscopes, oral and nasal pharyngeal airways, laryngeal masks, or para-esophageal airways may be inserted blindly (without the aid of a laryngoscope) to assist delivery of oxygen to the patient.

Most of these devices do not incorporate a video display, but transmit the distal image to an optical eyepiece, which requires positioning the intubator close to the patents mouth where potential exposure to vomitus or other secretions may occur. A separate video camera can often be attached to these devices. The use of a video screen rather than an optical eyepiece is advantageous to allow simultaneous viewing of both the internal and external condition of the patient. These separate light source and video cameras and displays also require separate focusing, control boxes, AC power cords and convenient AC outlets for use. This prohibits use in field conditions which might be found by EMT or ambulance responders.

However, none of these devices, by themselves, enter the trachea. Therefore, such devices may not provide a permanent solution for intubation of the trachea which is necessary for ventilation of the patient, the prevention of aspiration, respiratory therapy or certain surgical procedures. These devices all have fixed curves and endotracheal tube guide paths which may not be suitable for all patients.

Devices designed to enter the trachea are disclosed in the prior art, but do not adequately solve all the problems.

George, U.S. Pat. No. 4,742,819 discloses a semi-malleable rigid stylet placed inside the endotracheal tube, with an external video screen. An alternative embodiment described utilizes a lever to manipulate the distal end of the device, but its dependence on the pre-formed curve of the remaining stylet may be inadequate for all anatomic variations encountered. Also fiber optic bundles may break with repeated use, degrading the video signal.

Greene, U.S. Pat. No. 5,327,881 discloses a rigid fiber optic intubating stylet with distal acute angle tip angulation. This device suffers from fiber optic bundle degradation and well as non-anatomic shaping of the stylet portion. Having an eyepiece only, it lacks video display, forcing the user to place their eye in proximity of exposure to patient expectorations.

Flarn, U.S. Pat. No. 5,607,386 discloses an apparatus to guide fiber optic bronchoscopes with mounted endotracheal tubes into the trachea, but suffers from the use of fiber optics, and the bronchoscope with conduits must be taken out of service for time-consuming cleaning and sterilization. The device is large and cumbersome in use as depicted in the drawings.

Raybum et al, U.S. Pat. No. 5,733,242 discloses an intubation system which can be located in the trachea and an endotracheal tube advanced over it into position. This device lacks a video display and suffers from fiber optics and a pre-bent shape which is not adjustable during use, except for a short tip portion similar to George, above, with a lens that needs to be treated to prevent fogging. The presence of conduits require formal cleaning and sterilization.

Bashour, U.S. Pat. Nos. 5,803,898 and 6,432,042 discloses an ‘endoscopic stylet’ which is semi rigid, and requires the user to ‘anticipate the airway contours’ in that the device lever only steers the distal tip. This device suffers not only from fiber optics and the lack of a video display, but also must be pre-formed into what might be the correct shape for the individual patient, and must be withdrawn, re-shaped and reinserted. It has no ergonomic handle, and the stylet with the mounted endotracheal tube must be awkwardly held directly.

Sanders et al. U.S. Pat. No. 5,913,816 discloses an intubation device and method which describes in general terms the theory of every endoscopic intubation device. The disclosed device needs separate light and camera sources and monitors, as well as utilizing conduits which dictate formal cleaning and sterilization procedures. Anatomically shaped steering is not described.

Nakaichi et al, U.S. Pat. Nos. 6,004,263, 6,319,195 and 6,432,043 discloses an endoscope for intubating having a bendable stylet element, but it only deflects in one direction, and may not be suitable for all patients. It also suffers from the utilization of fiber optics.

Schwartrz et al, U.S. Pat. No. 6,539,942 discloses an endotracheal intubation device bendable during use into an L-shaped configuration at the distal end to promote visualization of the vocal cords. Since the anatomic path of a properly placed endotracheal tube has never been described as ‘L-shaped’, it is not clear just exactly how this device could facilitate intubation. This device must also be used with a separate ‘scope’ device.

Chhibber et al, U.S. Pat. No. 6,832,986 discloses an endoscopic intubation system which is not bendable or adjustable, and designed specifically for newborn babies and suffers from fiber optic bundle effects.

Hill, U.S. Pat. No. 6,929,600 discloses a semi-malleable rigid stylet placed inside the endotracheal tube. with an external video screen. But the stylet is not adjustable during use, and must be withdrawn from the patient, readjusted, and the procedure reattempted if the initial user adjustment is not suitable for that particular patient. This device does not describe a handle and the stylet with the mounted endotracheal tube must be awkwardly held directly. Withdrawing this semi-rigid device along the length of the endotracheal tube after accomplishing intubation can also be difficult, as curve angles typically differ along the length of the tube, sometimes dislodging the endotracheal tube from its proper position. It also has conduits that require formal cleaning and sterilization.

Prior art devices are problematic using rigid or malleable stylets; not adjustable to guide ETT during intubation procedure, or provide tip flex (only), and cannot act as a stylet, only a guide using a two-part procedure. Prior art devices require complex, time consuming sterilization reprocessing.

The present invention is designed to overcome the disadvantages of the prior art, which are rigid or malleable and not adjustable to guide the endotracheal tube during the intubation procedure, or provide for tip flex only, and cannot act as a stylet, requiring a two-step procedure of first placing the device into the trachea and then using their device as a guide to advance the endotracheal tube into the trachea. The present invention allows a one-step intubation procedure intubating stylet designed to navigate airway anatomy with an anatomically correct insertion path which is adjustable during use. The present invention with its ergonomic handle steers and bends a conventional endotracheal tube into anatomic pathway shape during use and is stiff enough to displace intervening tissues (e.g. tongue, epiglottis) which may obscure the glottis during the intubation procedure, combining several desirable objectives in achieving successful endotracheal intubation.

### BRIEF SUMMARY OF THE INVENTION

It is a primary object of the present invention to provide a device which provides ease of use for any intubator even in awkward patient positions (e.g. car wreck, during neurosurgery, etc.) to easily guide an endotracheal tube and steer and shape the tube during insertion to conform to the actual shapes of the patient air passageways and utilizes a camera system mounted on the device with a viewing screen on the handle to observe the path of the intubation tube simultaneously with the observation of the external condition of the patient, and an ergonomically shaped handle which optimizes manipulation of the invention, providing means for steering the intubation tube during insertion.

The present invention overcomes all of the inadequacies of the intubation devices seen in the prior art. It can be used in the conscious or unconscious (anesthetized) patient, orally or nasally. It is adjustable during use to conform to the individual anatomy of the patient, the anatomic shaping by trigger lever promoting endotracheal tube placement without head, neck or jaw manipulation. The prefocused integral video display eliminates the need to place the intubators' head near the patients mouth with exposure to vomitus or other secretions. There are no fiber optical light transmission bundles to break with image degradation. The battery powered embodiment does not require power cords or AC availability for use. It is designed not to come into contact with mucus membranes and is further protected from cross contamination by use of the polymer sheath or condom. Simple construction with available technology provides easy affordability. It is meant to be used for all endotracheal intubation procedures to allow familiarity with use when difficult situations arise unexpectedly.

The intubation device of the present invention provides a tool useful for intubation procedures where usual methods can fail. This device can not only be used for endotracheal intubation procedures, but different embodiments can be used for specialized intubations, or assessment and surgery of any accessible body cavity.

It is an objective of the present invention to provide a device for intubation which is easy to use and readily available without the delay or expertise inherent in the use of fiber optic bronchoscopes or direct or video laryngoscopes.

It is a further objective to provide an intubation device which can be used orally or nasally.

It is a further objective to provide an intubation device with a self contained power source.

It is a further objective of the present invention to visually facilitate intubation using a tip located camera that does not utilize fiber optics, allowing observation of the internal and external condition of the patient.

It is a further objective to manually steer placement of the endotracheal tube by changing stylet shape to conform to the range of anatomical shapes necessary to navigate the entrances to the trachea in a variety of patients' individual situations, and then allow flaccid withdrawal of the stylet without dislodging the endotracheal tube.

It is a further objective to allow intubation of patients whose anatomy or clinical condition precludes direct laryngoscopy or other routine methods of intubation, and to accomplish intubation in situations where direct laryngoscopy is impossible or contraindicated.

A yet further object is to combine and improve upon aspects of prior endoscopic stylet video intubation systems to create a unique instrument incorporating the strengths of these systems and eliminating the weaknesses of the aforementioned systems.

It is a further objective to provide an intubation device in which the stylet portion is sealed, without conduits, and does not come into contact with patient mucus membranes, which uses a disposable cover or condom to insure sterility and prevent cross contamination between patients and allow rapid reuse for the next patient.

In brief, the preferred embodiment of the present invention relates to a system for inserting a surgical tube into an externally accessible passageway, such as intubating an endotracheal tube into the trachea of a patient. More particularly the present invention relates to a steerable video intubation device, comprising an ergonomic handle with video display and trigger lever adjustor controlling a flexible stylet containing steering and stiffening tendons, and a distal tip lens—camera—light source. Patient protection from cross contamination is provided by a disposable cover. An endotracheal tube is then slid over the covered stylet and secured to the endotracheal tube stop. The intubation device with mounted endotracheal tube uses its anatomic shaping, mechanical steering and video capabilities to allow insertion of the tube into the patients' trachea, at which time the device is withdrawn, leaving the endotracheal tube in the trachea.

The present invention comprises an intubating stylet configured to navigate airway anatomy anatomically correctly, to be ergonomically efficient and adjustable during use for steering and bending a conventional surgical tube into anatomic pathway shape during use, the stylet being stiff enough to displace intervening tissues (e.g. tongue, epiglottis) obscuring the glottis.

The intubation device of the present invention provides ease of use for any intubator even in awkward patient positions (e.g. car wreck, during neurosurgery, etc.) for an easily guided naturally bending endotracheal tube with conforms to the actual anatomic shapes of the patient passageways and utilizes a camera system mounted on the device with a viewing screen on the handle to observe the path of the intubation tube simultaneously with the observation of the external condition of the patient, and an ergonomically shaped handle which optimizes manipulation of the invention.

This and other uses of the invention will be become more obvious through the consideration of the drawings of preferred embodiments. Any accessible body cavity such as lungs, stomach, vagina, anus and rectum can be intubated or examined with different embodiments of the device.

### BRIEF DESCRIPTION OF THE SEVERAL VIEWS OF THE DRAWINGS

These and other details of the present invention will be described in connection with the accompanying drawings, which are furnished only by way of illustration and not in limitation of the invention, and in which drawings:

FIG. 1A is a side elevational view of an intubation device in accordance with the present invention, showing an impermeable transparent sheath used to cover the stylet to isolate the stylet portion of the present invention;

FIG. 1B is a side elevational view of the stylet portion of the intubation device of FIG. 1, showing the impermeable transparent sheath covering the stylet to isolate the stylet portion of the present invention;

FIG. 2A is a side elevational view in partial section of the intubation device of FIG. 1, demonstrating the lever controlled tendon mechanism to deflect, bend and firm the stylet;

FIG. 2B is an enlarged cross sectional view of the stylet portion of the intubation device taken at 2′-2′ of FIG. 2A, showing tendon race/guide channels for the anterior tendon and posterior tendon, respectively, for steering and stiffening the stylet, and a central channel for wiring from the camera to the screen;

FIG. 2C is a distal end view of distal face of the stylet of FIG. 2A showing the camera lens, light source, and heating element;

FIG. 3 is a side elevational view of an intubation device in accordance with the present invention, showing an impermeable transparent sheath covering the stylet and a tracheal tube into which the intubation stylet will be inserted to install the tracheal tube in the externally accessible interior passageway of the patient;

FIG. 4 is a side elevational view of an intubation device in accordance with the present invention, showing an impermeable transparent sheath covering the stylet and a tracheal tube covering the transparent sheath and the intubation stylet will be inserted to install the tracheal tube in the externally accessible interior passageway of the patient.

### DETAILED DESCRIPTION OF THE INVENTION

In FIGS. 1-4, a passageway conforming intubation system comprises a self-contained guided intubation device 110 for intubating passageways of patients. Referring to FIG. 1, the intubation device 110 comprises an ergonomically shaped handle 112 extending from a proximal end of an intubation device body 111. The body 111 may be positioned in different orientations depending upon the body location of the intubation procedure and the shape of the body and handle, but is shown in a horizontal orientation for an endotracheal intubation procedure. The handle 112 is angled downwardly away from a proximal end of the intubation device body 111 and a stylet 124 extending downwardly from a distal end. The handle 112 is ergonomically formed to fit the users hand and may be pistol shaped (depicted in FIG. 1) or fusiform, or based on a mold taken of the human hand. The handle 112 is preferably made from medical grade material and may be constructed from metal, such as aluminum, or high quality plastic or other polymer.

The stylet 124 is made from a medical grade bendable, compressible polymer. The stylet 124 extends downwardly from a downwardly angled distal portion 113 of the intubation device body 111 (depicted in a horizontal orientation) to provide user ergonomics during the endtracheal intubation procedure, as the typical entrance to a patients' mouth is vertically oriented when the patient is supine, which is the usual position for an endotracheal intubation procedure. A lever 116 extends downwardly at an angled from the intubation device body 111 at the intersection with the handle 112. The lever 116 preferably acts as a time limited on-off switch activating the light source and video display. The lever 116 also causes bending and extension of the stylet 124. The stylet 124 bends along its entire length approximating curves of progressively smaller radiuses. The range of the induced bend from the neutral position is preferably approximately 160 degrees to minus 30 degrees. The lever may be surrounded by a guard 118. A battery access opening with a cover 114 is provided at the handle bottom for accessing the rechargeable battery 214 in the handle, as shown in FIG. 2A.

A video display 134 preferably of the LED type, is attached to the intubation device body 111 by a pivot post 132, or built integral with, the intubation device body 111. The stylet 124 is sealed along its length. This distal tip 122 of the stylet is smooth with rounded edges to prevent laryngeal injury during the intubation procedure.

In FIG. 2C, the distal tip of the stylet 122 contains a video camera 120 with a prefocused lens 123 providing suitable focal length and depth of field for the intubation procedure, as well as a warming element 121 to heat the tip 122 to normal body temperature as an aid to prevent lens fogging. The camera light 125, lens 123, and warming element 121 are conventional and not further described. A defogging chemical may be wiped onto the lens 123 of the camera 120 prior to use for maintaining a clear lens instead of the warming element 121, or in addition to the warming element.

In FIG. 2B, the wires 233 from the camera 120, light 125, and heating element 121 extend through a central opening 234 in the stylet 124 up to the batteries 214 in the handle 112.

In FIG. 4, a friction stop 126 is preferably used to secure a conventional endotracheal tube 500 mounting end 512 mounted with a friction fit over the friction stop 126 with the endotracheal tube covering the stylet 124. The endotracheal tube 500 is easily released from the stylet 124 when the endotracheal tube 500 is in place within the patient. The friction stop 126 is adjustable along the length of the stylet 124 and is secured to the stylet shaft either frictionally, by clamp, or cam lock, or other conventional means.

In FIGS. 1B, 3, and 4, the elasticized proximal end 426 of the impermeable transparent sheath 410 fits over the top of the friction stop 126 with a tight friction fit to maintain the impermeable transparent sheath over the stylet 124 during the entire procedure to maintain the sterility of the stylet. At least the distal tip 420 of the impermeable transparent sheath in transparent to permit clear visibility for use of the camera 120 and light 125.

Referring to FIG. 2A, the intubation device inner mechanism is shown in partial cross-sectional view. The battery 214 is contained within the handle 112. Demonstrated is the method by which the lever 116 imparts bending and stiffening to the flexible stylet 124. Manipulation of the lever 116 rotates a spool 212 which may be round (depicted), clam, or cam shaped. At least one tendon, but preferably two tendons 224 and 230, are advanced into or retracted from, the stylet, affecting its shape and stiffness. Depicted are two tendons, where a first tendon 224 acts to cause downward or posterior deflection, while a second tendon 230 controls upward or anterior deflection of the stylet 124. The course of these tendons from the lengthening/shortening spool 212 passes over a proximal, large guide 220, rotates up to 90 degrees, and then a over a distal, small guide 226 for proper orientation in the stylet shaft. The tendons are anchored to anchor points 218 at the distal end of the tendon race/guide way channels 238 and 232. The stylet 124 bends smoothly along its length approximating curves of progressively smaller radiuses. The range of the induced bend from the neutral position is approximately 160 degrees to minus 30 degrees.

In FIG. 2B the tendon race/guide way channels 232 and 238 allow movement of the anterior 230 and posterior 224 steering and stiffening tendons, respectively. A central channel 234 for wiring 233 is depicted, but the wiring can alternately be molded into the substance of the stylet shaft at time of manufacture.

In use, a tracheal tube is a catheter that is inserted into the trachea for the primary purpose of establishing and maintaining a patent airway and to ensure the adequate exchange of oxygen and carbon dioxide.

Many different types of tracheal tubes are available, suited for different specific applications. An endotracheal tube is a specific type of tracheal tube that is nearly always inserted through the mouth (orotracheal) or nose (nasotracheal).

### Operation

### Preferred Embodiment

In use with a conventional endotracheal tube 500, the user places a sterile cover, the impermeable transparent sheath 410, over the stylet 124 and a conventional endotracheal tube 500 is mounted on the covered stylet 124 and frictionally secured to the endotracheal tube stop 126 via its universal connector 512 with grasping plate 511. The endotracheal tube 500 is positioned on the stylet 124 such that the stylet tip 122 is almost flush, but not protruding from the distal end 520 of the endotracheal tube. The trigger lever 116 activates the light source and camera 120, and video display 134 for a predetermined period of time. The video display 134 may also indicate battery charge status and/or elapsed time from activation.

During use, the trigger lever 116 adjusts the stylet 124 by jamming or tensioning the tendons 224 and 230, shaping the stylet 124 into an anatomically correct curve. One tendon 224 acts to cause downward or posterior deflection, while a second tendon 230 controls upward or anterior deflection of the stylet 124. Stiffness is imparted by the tensioning and jamming of the tendons in their raceways 232 and 238 (FIG. 2B) resisted by the preformed shape of a conventional endotracheal tube. The stylet 124 bends smoothly along its length approximating curves of progressively smaller radiuses. The range of the induced bend from the neutral position is approximately 160 degrees up or anteriorly, to minus 30 degrees down or posteriorly. Thus the stylet can assume a range of anatomic shapes necessary to navigate the airway passages. The amount of force applied to the lever controls the amount of bend and inducement of curves of progressively smaller radius along the stylet. The device with endotracheal tube mounted thereon is introduced into the patient's hypopharynx, orally or nasally. The camera 120 and lens 123 focal length and depth of field are preset to display anatomic structures encountered during the intubation procedure. In the unconscious patient unable to maintain an open airway, a conventional direct laryngoscope or tongue blade may be used to lift the tongue and epiglottis out of the way. Using overall manipulation of the stylet and steering mechanism, the device with mounted endotracheal tube is advanced past the epiglottis until the vocal cords are identified videographically.

The device with mounted endotracheal tube is advanced through the vocal cords, thus positioning the endotracheal tube in the trachea, also videographically identified by its typical appearance. The balloon 521 at the distal end of the endotracheal tube 500 is manually inflated by the small pump 514 and air tube 513 to block the passageway except for the opening in the endotracheal tube 500 and the endotracheal tube 500 is then disconnected from the stylet 124 of the intubation device 110 while the stylet 124 is withdrawn, leaving the endotracheal tube in the trachea. The impermeable transparent sheath 410 is removed and discarded.

Additional Embodiment Pediatric and neonatal endotracheal tubes are much smaller and shorter than adult endotracheal tubes, and require a smaller sizing of the stylet 124 portion of the intubation device.

An intubation device to aid the placement of endobronchial tubes for separate lung ventilation and one lung anesthesia requires the stylet 124 portion to be longer.

An intubation device to aid other body cavity endoscopy. intubation, or surgery such as ENT diagnostic. gastroscopic, or proctoscopic model, can be utilized with protection of the device by a impermeable sheath or condom 410 for protecting alternate embodiments of the intubation device during procedures when the stylet may come into contact with sterile body cavities. The length of the stylet 124 portion, bend angles and induced curve radius would vary accordingly for these specialized uses.

Additional Embodiment. The operation of additional embodiments described above is essentially the same as that described for the preferred embodiment used for endotracheal intubation. Other body cavities would be approached by the appropriate external orifice and the protective sheath 410 of FIG. 1 used with the intubation device if sterile tissues are expected to be encountered.

Most endotracheal tubes today are constructed of polyvinyl chloride, but specialty tubes constructed of silicone rubber, latex rubber, or stainless steel are also widely available. Most tubes have an inflatable cuff 521 to seal the trachea and bronchial tree against air leakage and aspiration of gastric contents, blood, secretions, and other fluids. Uncuffed tubes are also available, though their use is limited mostly to pediatric patients (in small children, the cricoid cartilage, the narrowest portion of the pediatric airway, often provides an adequate seal for mechanical ventilation).

A Carlens double-lumen endotracheal tube is commonly used for thoracic surgical operations such as VATS lobectomy.

Types of endotracheal tube include oral or nasal, cuffed or uncuffed, preformed (e.g. RAE (Ring, Adair, and Elwyn) tube), reinforced tubes, and double-lumen endobronchial tubes. For human use, tubes range in size from 2 to 10.5 mm in internal diameter (ID). The size is chosen based on the patient's body size, with the smaller sizes being used for pediatric and neonatal patients. Tubes larger than 6 mm ID usually have an inflatable cuff. Originally made from red rubber, most modern tubes are made from polyvinyl chloride. Those placed in a laser field may be flexometallic. Robertshaw (and others) developed double-lumen endo-bronchial tubes for Thoracic surgery. These allow single-lung ventilation while the other lung is collapsed to make surgery easier. The deflated lung is re-inflated as surgery finishes to check for fistulas (tears).

The “armored” endotracheal tubes are cuffed, wire-reinforced, silicone rubber tubes which are quite flexible but yet difficult to compress or kink. This can make them useful for situations in which the trachea is anticipated to remain intubated for a prolonged duration, or if the neck is to remain flexed during surgery. Polyvinyl chloride tubes are relatively stiff in comparison. Preformed tubes (such as the oral and nasal RAE tubes, named after the inventors Ring, Adair and Elwyn) are also widely available for special applications. These may also be constructed of polyvinyl chloride or wire-reinforced silicone rubber. Other tubes (such as the Bivona Fome-Cuf tube) are designed specifically for use in laser surgery in and around the airway. Various types of double-lumen endotracheal (actually, endobronchial) tubes have been developed for ventilating each lung independently—this is useful during pulmonary and other thoracic operations.

The present invention is designed to overcome the disadvantages of the prior art, which are rigid or malleable and not adjustable to guide the endotracheal tube during the intubation procedure, or provide for tip flex only, and cannot act as a stylet, requiring a two-step procedure of first placing the device into the trachea and then using their device as a guide to advance the endotracheal tube into the trachea. The present invention allows a one-step intubation procedure intubating stylet designed to navigate airway anatomy with an anatomically correct insertion path which is adjustable during use. The present invention with its ergonomic handle steers and bends a conventional endotracheal tube into anatomic pathway shape during use and is stiff enough to displace intervening tissues (e.g. tongue, epiglottis) which may obscure the glottis during the intubation procedure, combining several desirable objectives in achieving successful endotracheal intubation.

Tracheal tubes can also be used to deliver oxygen in higher concentrations than found in air, or to administer other gases such as helium, nitric oxide, nitrous oxide, xenon, or certain volatile anesthetic agents such as desflurane, isoflurane, or sevoflurane. Tracheal tubes may also be used as a route for administration of certain medications such as salbutamol, atropine, epinephrine, ipratropium, and lidocaine. Tracheal tubes are commonly used for airway management in the settings of general anesthesia, critical care, mechanical ventilation, and emergency medicine.

It is understood that the preceding description is given merely by way of illustration and not in limitation of the invention and that various modifications may be made thereto without departing from the spirit of the invention as claimed.

What is claimed is:

1. A self-contained intubating stylet device to navigate airway anatomy anatomically correctly for intubating and adjusting tubular configuration during an intubation process into an externally accessible passageway of a patient, the device comprising in combination:
an intubation device body housing and supporting various components of the device;
an ergonomically shaped handle extending downwardly and angled away from a proximal end of the body; the handle structured in an ergonomic shape to fit a hand of a user to enable comfortable and effective use of the device;
an elongated configuration adjustable stylet attached to the body and extending vertically downward therefrom, the stylet removably insertable inside an entire length of a standard existing hollow flexible surgical tube, the stylet used for steering and guiding the surgical tube during insertion of the surgical tube into an externally accessable passageway of a patient, the stylet altering the configuration of the surgical tube during insertion to conform to the configuration of the passageway to enable the surgical tube to slide easily into the passageway and fit within the passageway of the patient after insertion and removal of the stylet to leave an interior elongated opening the length of the surgical tube as a clear airway in the passageway of the patient; the stylet comprising an elongated array of flexing, extending and stiffening tendons within the stylet along the length of the stylet, the tendons movable in cascading arrays so that the stylet bends along its entire length approximating curves of progressively smaller radiuses to enable the stylet to conform to the anatomically accurate actual shape of the passageway of the patient so that the stylet travels an anatomically accurate path into the internal passageway of the patient;
a control lever protruding from a bottom of the body adjacent to the handle in communication with the fingers of the user grasping the handle, the control lever used during insertion of the stylet, the control lever movable in a forward and backward relative to the handle by the fingers of the user operating the stylet, to steer the shape-transformable and steerable stylet through the passageway of the patient, the control lever adapted to transform the configuration of the stylet and the tracheal tube to conform to the configuration of the passageway;
a lens, a camera, and a light source located at a distal tip of the stylet to transmit a real time video image of the view in front of the stylet tip as the stylet is inserted into the passageways of the patient;
a power source communicating with the camera and light source;
a video display attached to the body, the video display in communication with the camera to receive the real time video image from the camera, the video display visible to the user holding the handle, the user simultaneously having visual communication with the exterior of the patient at the location of the insertion of the stylet so that the device provides a steerable video monitored intubation device with a surgical tube releasably mounted on a stylet which stylet is anatomically accurately shaped in real time during the intubation procedure using mechanical steering and video capabilities to allow insertion of the surgical tube into the passageway of the patient to a desired point and withdrawal of the stylet leaving the surgical tube in place in the passageway of the patient, thereby providing a self-contained guided intubation device for intubating the externally accessible passageways of patients.

2. The intubation device of claim 1 wherein the adjustment lever is connected to the body by a pivot wheel having a first cable attached from a first side of the pivot wheel to steering and stiffening tendons in the flexible stylet and a second cable attached from a second side of the pivot wheel to steering and stiffening tendons in the flexible stylet so that the stylet is adjustable to conform to the shape of the passageway of the patient by controlling the adjustment lever.

3. The intubation device of claim 2, wherein the flexible condition of the stylet is variably stiffened and curved into a range of anatomic shapes suitable to guide an external surgical tube from outside a patient, through an externally accessible passageway, and is adjustable during use.

4. The intubation device of claim 1 further comprising at least one warming element in the distal tip of the stylet to defog the camera lens, the at least one warming element powered by the power source.

5. The intubation device of claim 1 further comprising a defogging solution wiped on the camera lens to maintain clear viewing through the lens.

6. The intubation device of claim 1 wherein the video display displays real time visual landmarks of airway anatomy viewed by the camera.

7. The intubation device of claim 1 wherein the surgical tube comprises an endotracheal tube and the intubation device is used to guide an external endotracheal tube from outside a patient, through the nasopharynx or oropharynx and through the vocal cords into the trachea, and is adjustable during use.

8. The intubation device of claim 1 wherein the surgical tube intubated by the intubation device of the present invention comprises at least one type of surgical tube taken from the list of types of surgical tubes including an orotracheal endotracheal tube inserted through the mouth, nasotracheal endotracheal tube inserted through the nose, an inflatable cuff endotracheal tube for an adult patient, an uncuffed endotracheal tube for a pediatric patient, a polyvinyl chloride endotracheal tube, a silicone rubber endotracheal tube, a latex rubber endotracheal tube, a Carlens double-lumen endotracheal tube for thoracic surgical operations such as VATS lobectomy, a preformed endotracheal tube, a reinforced endotracheal tube, a double-lumen endobronchial tube, a flexometallic endotracheal tube for placement in a laser field, a Robertshaw double-lumen endo-bronchial tube for Thoracic surgery, a cuffed wire-reinforced silicone rubber armored endotracheal tube for a trachea anticipated to remain intubated for a prolonged duration and for the neck to remain flexed during surgery, a Bivona Fome-Cuf tube for use in laser surgery in and around the airway, a double-lumen endotracheal (endobronchial) tube for ventilating each lung independently during pulmonary and other thoracic operations.

9. The intubation device of claim 1 wherein the power source is housed in the handle.

10. The intubation device of claim 1 wherein the power source comprises disposable batteries.

11. The intubation device of claim 1 wherein the power source comprises rechargeable batteries.
